(19) United States
(12) Patent Application Publication
(10) Pub. No.: US 2026/0157640 A1
(43) Pub. Date: Jun. 11, 2026

(54) DETERMINING EXERCISE/FITNESS METRIC(S) AND CHANGE IN HEART RATE BASED ON MACHINE LEARNING

(71) Applicant: Apple Inc., Cupertino, CA (US)

(72) Inventors: Thilaka S. Sumanaweera, San Jose, CA (US); Gopal Valsan, Gilroy, CA (US); Jeffrey J. Richard, Menlo Park, CA (US); Justin P. Dobson, Sunnyvale, CA (US)

(21) Appl. No.: 19/323,861

(22) Filed: Sep. 9, 2025

Related U.S. Application Data
(60) Provisional application No. 63/692,685, filed on Sep. 9, 2024.

(51) Int. Cl. A61B 5/0205; A61B 5/00; A61B 5/024; A61B 5/11; G06T 7/00; G06T 7/70; G16H 20/30

(52) U.S. Cl. CPC A61B 5/0205; A61B 5/1128; A61B 5/4869; A61B 5/7267; G06T 7/0016; G06T 7/70; G16H 20/30; A61B 5/02405; G06T 2207/10016; G06T 2207/20081; G06T 2207/20084; G06T 2207/30196

(57) ABSTRACT

Embodiments are disclosed for determining at least one exercise or fitness metric and change in heart rate based muscle mass and machine learning. In some embodiments, a method comprises: capturing, with at least one camera, images of a user's body; receiving, with at least one processor, the user's weight; obtaining, with the at least one processor, the user's body composition based on the images and weight or from a previously stored user body composition from another source; determining, with the at least one processor, the user's muscle mass based on the determined body composition of the user; and determining, with a machine learning model, at least one exercise or fitness metric of the user during exercise based on the muscle mass of the user and the user's body motion during exercise. In some embodiments, change of heart rate is also tracked.

### CROSS-REFERENCE TO RELATED APPLICATION

This application claims priority U.S. Provisional Patent Application No. 63/692,685, filed Sep. 9, 2024, the entire contents of which are incorporated herein by reference.

### TECHNICAL FIELD

This disclosure relates generally to determining exercise/fitness metric(s) such as calorie consumption and change in heart rate during exercise.

### BACKGROUND

Existing methods for estimating calorie consumption and change in heart rate assume every kilogram of the human body contributes equally to energy metabolism. The human body, however, comprises many different types of tissue, such as muscle, adipose tissue (fat), bone, cartilage, etc. These different tissue types lead to inaccurate estimation of calorie consumption using current calorie consumption estimation methods that do not distinguish between the different tissue types. Moreover, since a change in heart rate from a resting or baseline heartrate during exercise is also related to energy metabolism, the accuracy of estimating the change of heart rate also suffers when using these existing methods.

### SUMMARY

Embodiments are disclosed for determining at least one exercise or fitness metric and change in heart rate based on muscle mass and machine learning.

In some embodiments, a method comprises: capturing, with at least one camera, images of a user's body; receiving, with at least one processor, the user's weight; obtaining, with the at least one processor, the user's body composition based on the images and weight or a previously stored user body composition from another source; determining, with the at least one processor, the user's muscle mass based on the determined body composition of the user; and determining, with a machine learning model, at least one exercise or fitness metric of the user during exercise based on the muscle mass of the user and the user's body motion during exercise.

In some embodiments, the method further comprises: determining, with the machine learning model, a change in heart rate of the user during exercise based on the muscle mass of the user and the user's body motion during exercise.

In some embodiments, a first machine learning model determines the at least one exercise or fitness metric of the user and a second machine learning model determines the change in the user's heart rate.

In some embodiments, the machine learning model is trained on at least one of a gravity vector, body orientation, center of mass, or center of gravity.

In some embodiments, inputs into the machine learning model include segmental muscle mass.

In some embodiments, inputs into the machine learning model include segmental muscle mass and segmental total mass.

In some embodiments, a method comprises: capturing, with at least one camera, a first video of at least one subject exercising; capturing, with at least one processor, calorie consumption and heart rate of the at least one subject while exercising; determining, with a first processor, joint coordinates of the at least one subject based on the first video; obtaining, with the first processor, a body composition of the at least one subject based on the joint coordinates and weight of the at least one subject or previously stored user body composition from another source; training, with the first processor, at least one machine learning model, with the calorie consumption, heart rate and body composition of the at least one subject; receiving, with at least one processor, a second video and muscle mass of a user exercising; and determining, with the at least one processor, at least one exercise or fitness metric and change of heart rate of the user during exercise, where the determining is based on the at least one machine learning model, the second video and the user's muscle mass.

In some embodiments, the method further comprises: determining, at the end of a user workout, muscle fatigue by analyzing at least one of the first or second video and determining which muscles were used the most; and suggesting to the user a period of recovery for the muscles that were used the most.

In some embodiments, the muscle fatigue is quantified into a metric.

In some embodiments, the determined change in heart rate during a workout is used to estimate cardiovascular efficiency.

Other embodiments are directed to an apparatus, system and computer-readable medium.

Particular embodiments described herein provide one or more of the following advantages. Using machine learning models trained on muscle mass provides a more accurate estimate of calorie consumption and change of heart rate of a user during exercise than existing methods that do not distinguish between different tissue types.

### BRIEF DESCRIPTION OF THE DRAWINGS

FIG. 1 is a block diagram of a prior art system of estimating calorie consumption and change of heart rate of a user during exercise.

FIG. 2 is a block diagram of a system of estimating calorie consumption and change of heart rate of a user during exercise based on muscle mass, according to one or more embodiments.

FIG. 3 is a skeletal model illustrating segmental muscle mass distribution, according to one or more embodiments.

FIG. 4 is a block diagram of a system of estimating calorie consumption and change in heart rate of a user during exercise based on segmental muscle mass distribution, according to one or more embodiments.

FIG. 5 is a flow diagram of a process of estimating calorie consumption and change of heart rate of a user during exercise based on muscle mass, as described in reference to FIGS. 1-4.

FIG. 6 is a block diagram of a device architecture for implementing the features and processes described in reference to FIGS. 1-5.

### DETAILED DESCRIPTION

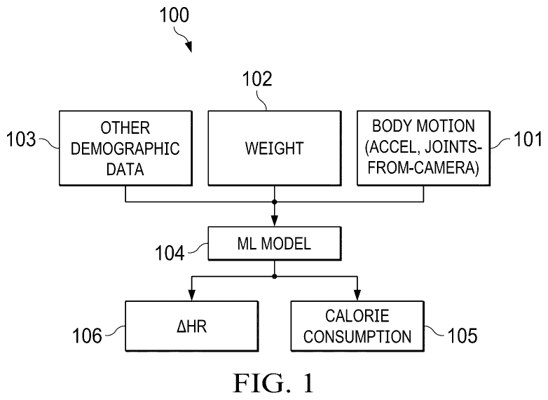

FIG. 1 is a block diagram of a prior art system of estimating calorie consumption and change of heart rate of a user during exercise. System 100 includes machine learning model 104 (e.g., a neural network) that receives as input body motion 101 of a user during exercise, weight 102 of the user and other demographic data 103 (e.g., age, gender). Machine learning model 104 is trained to estimate calorie consumption 105 and change in heart rate 106 of the user.

System 100 assumes every kilogram of the user's body contributes equally to the estimate of calorie consumption 105. The human body, however, comprises many different types of tissue, such as muscle, adipose tissue (fat), bone, cartilage, etc. These different tissue types lead to inaccurate estimation of calorie consumption 105. Since a change in the user's heart rate from a resting or baseline heartrate during exercise is also related to energy metabolism, the accuracy of the estimate the change of user's heart rate 106 is also in accurate.

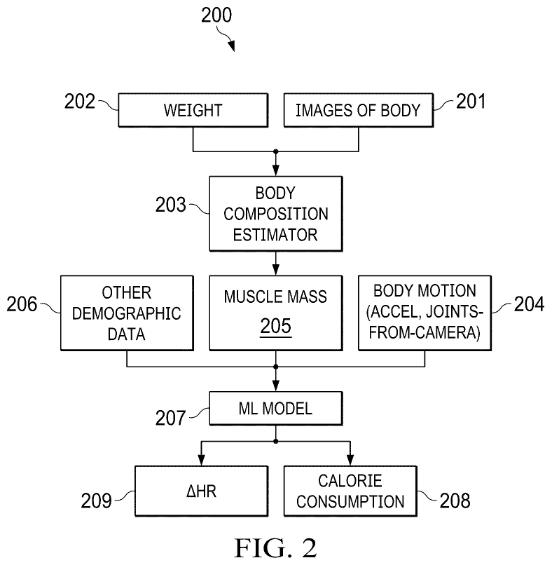

FIG. 2 is a block diagram of a system of estimating calorie consumption and change of heart rate of a user during exercise based on muscle mass, according to one or more embodiments. System 200 can be implemented on, for example, a smartphone, mobile computer, tablet computer or a wearable device, such as a smartwatch or head mounted device. System 200 includes body composition estimator 203 and machine learning model 207. Body composition estimator 203 takes as input images 201 of a user's body during exercise (e.g., captured by a video camera) and the user's total body weight 202 (e.g., entered by the user through a user interface). Body composition estimator 203 includes a body composition model that estimates a percentage of the user's total body weight 202 that comprises adipose-free tissue 205 or lean mass. In some embodiments, body composition can be obtained from health data stored in the device.

The user's muscle mass 205 and body motion 204 during exercise are input into at least one machine learning model 207, which estimates exercise or fitness metrics for the user, such as the user's calorie consumption 208 and change in heartrate (ΔHR) 209 relative to a baseline or resting heart rate. The user's body motion (e.g., acceleration) can be captured by inertial sensors (e.g., a 3-axis accelerometer) embedded on their smartphone, smartwatch or any other body worn device (e.g., fitness band). In some embodiments, body motion 204 includes three-dimensional (3D) joint coordinates determined from images 201 by a body tracking algorithm. An example body tracking algorithm is incorporated in ARKit™, which is available in the IOS 13+ operating systems developed by Apple Inc. of Cupertino, California USA. In some embodiments, the machine learning model can take as inputs environmental conditions, such as altitude, inertial measurements (e.g., from an inertial measurement unit (EIU)), air density, temperature, humidity, etc., to improve its estimation of different exercise or fitness metrics.

In some embodiments, machine learning model 207 is trained on images, calorie consumption, heart rate and optionally demographic features (e.g., age, gender) of subjects exercising. The subjects can be, e.g., walking on a treadmill. One or more cameras can be used to capture images (e.g., videos) of different angles of the user walking on the treadmill. These images and/or augmented versions of the images can be used to train machine learning model 207. In some embodiment, synthetic images using a skeletal model can be used to generate training images in addition or in lieu of the camera images.

Machine learning model 207 can be any suitable machine learning model, including but not limited to: artificial neural networks (e.g., deep learning networks), decision trees, support vector machines, Naïve Bayesian classifiers, Bayesian network, k-nearest neighbor, random forest transformers, recurrent networks or any other suitable machine learning model. In some embodiments, machine learning model 207 can include more than one machine learning model, such as a first model for estimating calorie consumption and a second model for estimating change in heartrate, where each model can be trained on the same or different data sets. If deep neural networks are used, then any suitable architecture can be used, including but not limited to any number or type of hidden layers, pooling layers or activation functions.

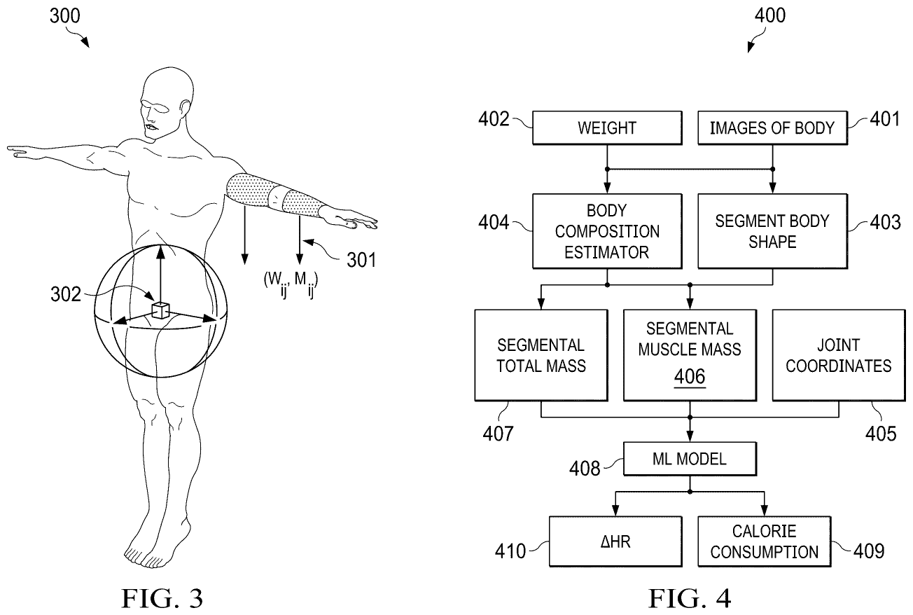

FIG. 3 is a skeletal model illustrating segmental muscle mass distribution calculations, according to one or more embodiments. Example skeletal model 300 is associated with a reference right-handed coordinate system with an origin at the pelvic region 302. Each segment is separated by two joints i and j that have joint coordinates (xₖ(t), yₖ(t), zₖ(t)) that change as a function of time. The joint coordinates can be estimated by a body tracking algorithm as described above. The estimated total mass Wij of a segment 301 between joints i and j is estimated, and the muscle mass in the same segment Mij is also estimated from images of the body and the user's total weight. These estimates can be used as additional inputs into a machine learning model to estimate calorie consumption 409 and change in heart rate 410, as described below in reference to FIG. 4.

In some embodiments, segmental mass and segmental muscle mass are determined by assuming the body is made of fat and muscle. Referring to FIG. 3, Let the total mass, total muscle mass and the total body volume be represented by the variables W, M and V, respectively.

The total body density is given by:

$${ρ}_{w} = \frac{W}{V} ,$$

the “effective” total body muscle density is given by:

and

$${ρ}_{m} = \frac{M}{V} ,$$

the volume of the segment between the link i and link j is Vij, where the segmental mass W_{i,j} and the segmental muscle mass M_{i,j} are given by:

$${W}_{i , j} = {ρ}_{w} {V}_{ij} , and$$

$${M}_{i , j} = {ρ}_{m} {V}_{ij} .$$

FIG. 4 is a block diagram of a system of estimating calorie consumption and change in heart rate of a user during exercise based on segmental muscle mass, according to one or more embodiments. Images 401 of the user's body and total weight 402 of the user are input into segment body shape estimator 403 and body composition estimator 404. The body composition estimator 404 determines the percentage of the total weight that is muscle mass as previously described in reference to FIG. 2. In some embodiments, the body composition is a previously stored body composition from another source (e.g., medical/health/fitness assessments). Segment body shape estimator 403 determines the shape (e.g., volume) of each limb from the images 401 of the body. The shape of the limb is used to estimate segmental muscle mass 406 and segmental total mass 407. Joint coordinates 405 computed from images 401, segmental muscle mass 406 and segmental total mass 407 are input into machine learning model 408, which is trained to determine 409 at least one exercise or fitness metric (e.g., the user's calorie consumption) for the user and change in the user's heart rate 410, as previously described.

In some embodiments, a deep learning artificial neural network (e.g., a U-net) is used for automatic segmentation of skeletal muscle segments. For example, in some embodiments an image based convolutional neural network (CNN) (e.g., ResNet) can be used to detect the limbs of the user in the video images. The two-dimensional detections are output from the CNN as 2D bounding boxes which are then transformed into 3D bounding boxes using geometry, intrinsic camera parameters and a pinhole model using techniques known in the art. The three dimensions of the bounding boxes are used to determine the volume of a limb contained in a 3D bounding box.

The percentage of muscle mass provided by body composition estimator 404 can be used with the known density of muscle tissue to estimate the segmental muscle mass of the limb (mass=(density/volume)×% of muscle mass). For example, a man's arm may be 4 kg total weight with 16% muscle mass. In some embodiments, if the device has a depth sensor, such as a time-of-flight sensor, the depth data from that sensor can be used to identify segments.

In some embodiments, a neural network for segmental body composition can be trained using MRI or CT/Xray/DEXA data. In some embodiments, ground truth data can include measurements of skin folds (segmental body fat) using a caliber or other suitable body fat measuring device. Some examples of skin folds that could be measured using a caliper include but are not limited to: sub-scapular, axilla, chest, abdomen, suprailia, thigh and triceps.

In some embodiments, regional body composition can be estimated using DEXA data as ground truth, such as described in Zheng Y, Long Z, Zhang X, Hahn JK. “3D Body shape for regional and appendicular body composition estimation.” Proc SPIE Int Soc. Opt. Eng. 2023 February; 12464:124642C. Doi: 10.1117/12.2653964. Epub. 2023 Apr. 3. PMID: 37456532; PMCID: PMC10348601.

In some embodiments, muscle fatigue is estimated at the end of a workout by analyzing the videos and looking at which muscles were “used” the most, and suggest to the user a period of recovery for those muscles.

In some embodiments, muscle fatigue can be quantified into a metric for trend tracking so that the user can implement more “balanced” training in their workout.

In some embodiments, the estimated change in heart rate during a workout is used to estimate cardiovascular efficiency, such as VO2 Max.

### Example Process

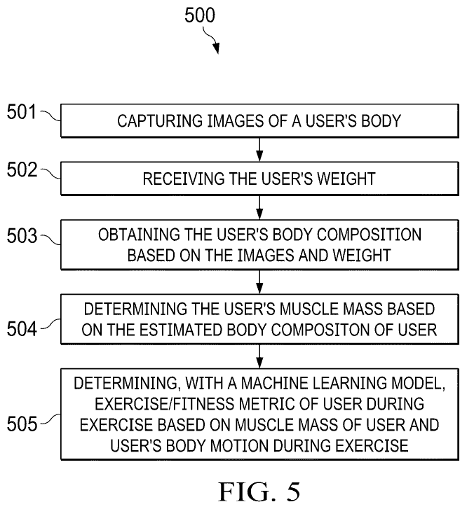

FIG. 5 is a flow diagram of a process 500 of estimating calorie consumption and change of heart rate of a user during exercise based on muscle mass, as described in reference to FIGS. 1-4. Process 500 includes: capturing, with at least one camera, images of a user's body (501); receiving, with at least one processor, the user's weight (502); obtaining, with the at least one processor, the user's body composition based on the images and weight, or from a previously stored body composition of the user from another source (503); determining, with the at least one processor, the user's muscle mass based on the estimated body composition of the user (504); and determining, with a machine learning model, at least one exercise or fitness metric of the user (e.g., calorie consumption) during exercise based on the muscle mass of the user and the user's body motion during exercise (505). Each of these steps were previously described in detail in reference to FIGS. 1-4. In some embodiments, the stored user body composition can be obtained from medical/health/fitness assessments. In some embodiments, a change in the user's heart rate is also estimated by the machine learning model. In some embodiments, confidence scores are generated by the machine learning model indicative of the confidence of the estimated calorie consumption and change in heartrate.

### Example Device Architecture

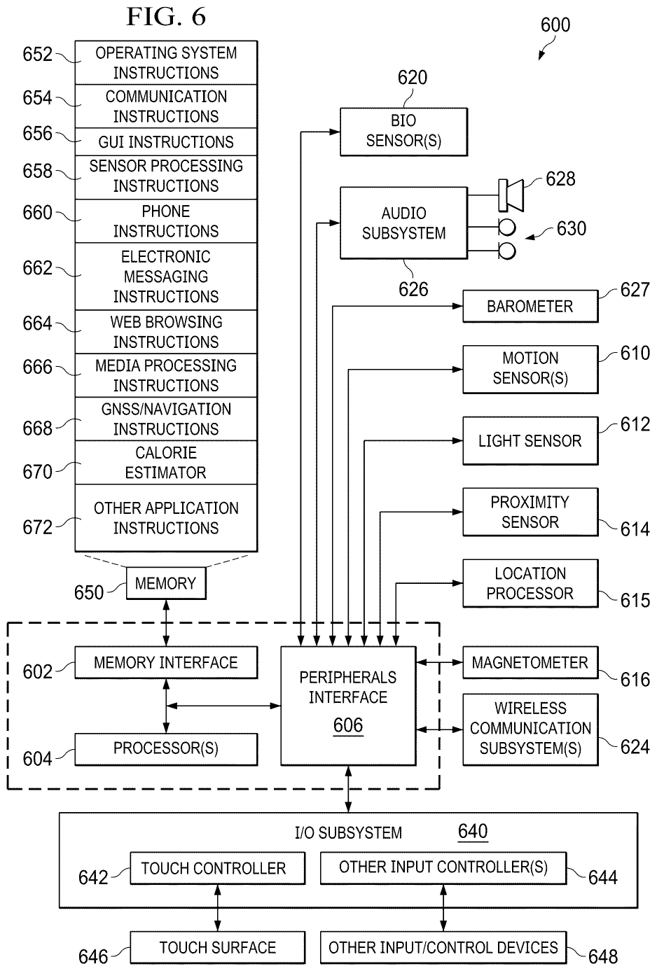

FIG. 6 is a block diagram of a crash device architecture 600 for implementing the features and processes described in reference to FIGS. 1-4. Architecture 600 can include memory interface 602, one or more hardware data processors, image processors and/or processors 604 and peripherals interface 606. Memory interface 602, one or more processors 604 and/or peripherals interface 606 can be separate components or can be integrated in one or more integrated circuits. System architecture 600 can be included in any suitable electronic device, including but not limited to: a smartwatch, smartphone, fitness band and any other device that can be attached, worn, or held by a user.

Sensors, devices, and subsystems can be coupled to peripherals interface 606 to provide multiple functionalities. For example, one or more motion sensors 610, light sensor 612 and proximity sensor 614 can be coupled to peripherals interface 606 to facilitate motion sensing (e.g., acceleration, rotation rates), lighting and proximity functions of the wearable device. Location processor 615 can be connected to peripherals interface 606 to provide geo-positioning. In some implementations, location processor 615 can be a GNSS receiver, such as the Global Positioning System (GPS) receiver. Electronic magnetometer 616 (e.g., an integrated circuit chip) can also be connected to peripherals interface 606 to provide data that can be used to determine the direction of magnetic North. Electronic magnetometer 616 can provide data to an electronic compass application. Motion sensor(s) 610 can include one or more accelerometers and/or gyros configured to determine change of speed and direction of movement. Barometer 617 can be configured to measure atmospheric pressure (e.g., pressure change inside a vehicle). Bio sensor(s) 620 can be one or more of a PPG sensor for measuring heart rate, an electroencephalogram (EEG) sensor, an electrocardiogram (ECG) sensor, an electromyogram (EMG) sensor, a mechanomyogram (MMG) sensor (e.g., piezo resistive sensor) for measuring muscle activity/contractions, an electrooculography (EOG) sensor, a galvanic skin response (GSR) sensor, a magnetoencephalogram (MEG) sensor and/or other suitable sensor(s) configured to measure bio signals.

Communication functions can be facilitated through wireless communication subsystems 624, which can include radio frequency (RF) receivers and transmitters (or transceivers) and/or optical (e.g., infrared) receivers and transmitters. The specific design and implementation of the communication subsystem 624 can depend on the communication network(s) over which a mobile device is intended to operate. For example, architecture 600 can include communication subsystems 624 designed to operate over a GSM network, a GPRS network, an EDGE network, a WiFi™ network and a Bluetooth™ network. In particular, the wireless communication subsystems 624 can include hosting protocols, such that the crash device can be configured as a base station for other wireless devices.

Audio subsystem 626 can be coupled to a speaker 628 and a microphone 30 to facilitate voice-enabled functions, such as voice recognition, voice replication, digital recording, and telephony functions. Audio subsystem 626 can be configured to receive voice commands from the user. Audio subsystem 626 can be used to capture audio and to convert the audio to SPL for additional processing.

I/O subsystem 640 can include touch surface controller 642 and/or other input controller(s) 644. Touch surface controller 642 can be coupled to a touch surface 646. Touch surface 646 and touch surface controller 642 can, for example, detect contact and movement or break thereof using any of a plurality of touch sensitivity technologies, including but not limited to capacitive, resistive, infrared, and surface acoustic wave technologies, as well as other proximity sensor arrays or other elements for determining one or more points of contact with touch surface 646. Touch surface 646 can include, for example, a touch screen or the digital crown of a smart watch. I/O subsystem 640 can include a haptic engine or device for providing haptic feedback (e.g., vibration) in response to commands from processor 604. In an embodiment, touch surface 646 can be a pressure-sensitive surface.

Other input controller(s) 644 can be coupled to other input/control devices 648, such as one or more buttons, rocker switches, thumbwheel, infrared port, and USB port. The one or more buttons (not shown) can include an up/down button for volume control of speaker 628 and/or microphone 630. Touch surface 646 or other controllers 644 (e.g., a button) can include, or be coupled to, fingerprint identification circuitry for use with a fingerprint authentication application to authenticate a user based on their fingerprint(s).

In one implementation, a pressing of the button for a first duration may disengage a lock of the touch surface 646; and a pressing of the button for a second duration that is longer than the first duration may turn power to the mobile device on or off. The user may be able to customize a functionality of one or more of the buttons. The touch surface 646 can, for example, also be used to implement virtual or soft buttons.

In some implementations, the mobile device can present recorded audio and/or video files, such as MP3, AAC and MPEG files. In some implementations, the mobile device can include the functionality of an MP3 player. Other input/output and control devices can also be used.

Memory interface 602 can be coupled to memory 650. Memory 650 can include high-speed random-access memory and/or non-volatile memory, such as one or more magnetic disk storage devices, one or more optical storage devices and/or flash memory (e.g., NAND, NOR). Memory 650 can store operating system 652, such as the iOS operating system developed by Apple Inc. of Cupertino, California. Operating system 652 may include instructions for handling basic system services and for performing hardware dependent tasks. In some implementations, operating system 652 can include a kernel (e.g., UNIX kernel).

Memory 650 may also store communication instructions 654 to facilitate communicating with one or more additional devices, one or more computers and/or one or more servers, such as, for example, instructions for implementing a software stack for wired or wireless communications with other devices. Memory 650 may include graphical user interface instructions 656 to facilitate graphic user interface processing; sensor processing instructions 658 to facilitate sensor-related processing and functions; phone instructions 660 to facilitate phone-related processes and functions; electronic messaging instructions 662 to facilitate electronic-messaging related processes and functions; web browsing instructions 664 to facilitate web browsing-related processes and functions; media processing instructions 666 to facilitate media processing-related processes and functions; GNSS/Location instructions 668 to facilitate generic GNSS and location-related processes and instructions; and calorie estimator instructions 670 that implement the processes described in reference to FIGS. 1-5. Memory 650 further includes other application instructions 672 including but not limited to instructions for applications that utilize crash detection output.

Each of the above identified instructions and applications can correspond to a set of instructions for performing one or more functions described above. These instructions need not be implemented as separate software programs, procedures, or modules. Memory 650 can include additional instructions or fewer instructions. Furthermore, various functions of the mobile device may be implemented in hardware and/or in software, including in one or more signal processing and/or application specific integrated circuits.

While this specification contains many specific implementation details, these should not be construed as limitations on the scope of any inventions or of what may be claimed, but rather as descriptions of features specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable sub combination. Moreover, although features may be described above as acting in certain combinations and even initially claimed as such, one or more features from a claimed combination can in some cases be excised from the combination, and the claimed combination may be directed to a sub combination or variation of a sub combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

As described above, some aspects of the subject matter of this specification include gathering and use of data available from various sources to improve services a mobile device can provide to a user. The present disclosure contemplates that in some instances, this gathered data may identify a particular location or an address based on device usage. Such personal information data can include location-based data, addresses, subscriber account identifiers, or other identifying information.

The present disclosure further contemplates that the entities responsible for the collection, analysis, disclosure, transfer, storage, or other use of such personal information data will comply with well-established privacy policies and/or privacy practices. In particular, such entities should implement and consistently use privacy policies and practices that are generally recognized as meeting or exceeding industry or governmental requirements for maintaining personal information data private and secure. For example, personal information from users should be collected for legitimate and reasonable uses of the entity and not shared or sold outside of those legitimate uses. Further, such collection should occur only after receiving the informed consent of the users. Additionally, such entities would take any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices.

In the case of advertisement delivery services, the present disclosure also contemplates embodiments in which users selectively block the use of, or access to, personal information data. That is, the present disclosure contemplates that hardware and/or software elements can be provided to prevent or block access to such personal information data. For example, in the case of advertisement delivery services, the present technology can be configured to allow users to select to “opt in” or “opt out” of participation in the collection of personal information data during registration for services.

Therefore, although the present disclosure broadly covers use of personal information data to implement one or more various disclosed embodiments, the present disclosure also contemplates that the various embodiments can also be implemented without the need for accessing such personal information data. That is, the various embodiments of the present technology are not rendered inoperable due to the lack of all or a portion of such personal information data. For example, content can be selected and delivered to users by inferring preferences based on non-personal information data or a bare minimum amount of personal information, such as the content being requested by the device associated with a user, other non-personal information available to the content delivery services, or publicly available information.

1. A method comprising:
capturing, with at least one camera, images of a user's body;
receiving, with at least one processor, the user's weight;
obtaining, with the at least one processor, the user's body composition based on the images and the user's weight or a percent volume or from a previously stored user body composition from another source;
determining, with the at least one processor, the user's muscle mass based on the estimated body composition of the user; and
determining, with a machine learning model, at least one exercise or fitness metric for the user based on the muscle mass of the user and the user's body motion during exercise.

2. The method of claim 1, further comprising:
determining, with the machine learning model, a change in heart rate of the user during exercise based on the muscle mass of the user and the user's body motion during exercise.

3. The method of claim 2, wherein a first machine learning model determines the at least one exercise or fitness metric for the user and a second machine learning model determines the change in the user's heart rate.

4. The method of claim 1, wherein the machine learning model is trained on at least one of a gravity vector, body orientation, center of mass, or center of gravity.

5. The method of claim 1, wherein inputs into the machine learning model include segmental muscle mass.

6. The method of claim 5, wherein inputs into the machine learning model include segmental muscle mass and segmental mass.

7. A method comprising:
capturing, with at least one camera, a first video of at least one subject exercising;
capturing, with at least one processor, exercise data and heart rate of the at least one subject while exercising;
determining, with a first processor, joint coordinates of the at least one subject based on the first video;
obtaining, with the first processor, a body composition of the at least one subject based on the joint coordinates and weight of the at least one subject or from a previously stored user body composition from another source;
training, with the first processor, at least one machine learning model, with the exercise data, heart rate and body composition of the at least one subject;
receiving, with at least one processor, a second video and muscle mass of a user exercising; and
determining, with the at least one processor, at least one exercise or fitness metric for the user and change of heart rate of the user during exercise, where the determining is based on the at least one machine learning model, the second video and the user's muscle mass.

8. The method of claim 7, wherein a first machine learning model determines the at least one exercise or fitness metric of the user and a second machine learning model determines the change in the user's heart rate.

9. The method of claim 7, wherein the at least one machine learning model is also trained on at least one of a gravity vector, body orientation, center of mass, or center of gravity.

10. The method of claim 7, wherein inputs into the at least one machine learning model includes segmental muscle mass and segmental mass.

11. A system comprising:
at least one camera;
at least one processor;
memory storing instructions that when executed by the at least one processor, cause the at least one processor to perform operations comprising:
capturing, with the at least one camera, images of a user's body;
receiving the user's weight;
obtaining the user's body composition based on the images and weight or from a previously stored user body composition from another source;
determining the user's muscle mass based on the body composition of the user; and
determining at least one exercise or fitness metric of the user during exercise based on the muscle mass of the user and the user's body motion during exercise.

12. The system of claim 11, further comprising:
determining, with the machine learning model, a change in heart rate of the user during exercise based on the muscle mass of the user and the user's body motion during exercise.

13. The system of claim 12, wherein a first machine learning model determines the at least one exercise or fitness metric of the user and a second machine learning model determines the change in the user's heart rate.

14. The system of claim 11, wherein the machine learning model is trained on at least one of a gravity vector, body orientation, center of mass, or center of gravity.

15. The system of claim 11, wherein inputs into the machine learning model include segmental muscle mass.

16. The system of claim 11, wherein inputs into the machine learning model include segmental muscle mass and segmental mass.

17. A system comprising:
at least one camera;
at least one processor;
memory storing instructions that when executed by the at least one processor, cause the at least one processor to perform operations comprising:
capturing, with the at least one camera, a first video of at least one subject exercising;
determining calorie consumption and heart rate of the at least one subject while exercising;
estimating joint coordinates of the at least one subject based on the first video;
obtaining a body composition of the at least one subject based on the joint coordinates and weight of the at least one subject or from a previously stored user body composition from another source;
training at least one machine learning model, with the calorie consumption, heart rate and body composition of the at least one subject;
receiving a second video and muscle mass of a user exercising; and
determining at least one exercise or fitness metric of the user and change in heart rate of the user during exercise, where the determining is based on the at least one machine learning model, the second video and the user's muscle mass.

18. The system of claim 17, wherein a first machine learning model determines the at least one exercise or fitness metric of the user and a second machine learning model determines the change in the user's heart rate.

19. The system of claim 17, wherein the at least one machine learning model is also trained on at least one of a gravity vector, body orientation, center of mass, or center of gravity.

20. The system of claim 17, wherein inputs into the at least one machine learning model includes segmental muscle mass and segmental mass.

21. The system of claim 17, further comprising:
determining, at the end of a user workout, muscle fatigue by analyzing at least one of the first or second video and determining which muscles were used the most; and
suggesting to the user a period of recovery for the muscles that were used the most.

22. The system of claim 21, wherein the muscle fatigue is quantified into a metric.

23. The system of claim 21, wherein the determined change in heart rate during a workout is used to estimate cardiovascular efficiency.
